(19) United States
(12) Patent Application Publication
(10) Pub. No.: US 2005/0090760 A1
(43) Pub. Date: Apr. 28, 2005

(54) Impedance measuring device and health care guideline advising device

(75) Inventor: Tatsuya Kobayashi, Kyoto-shi (JP)

Correspondence Address: Barry E. Bretschneider;Morrison & Foerster LLP, Suite 300, 1650 Tysons Boulevard, McLean, VA, 22102, US

(73) Assignee: OMRON HEALTHCARE Co., Ltd., Kyoto-shi (JP)

(21) Appl. No.: 10/969,848

(22) Filed: Oct. 22, 2004

(30) Foreign Application Priority Data
Oct. 24, 2003 (JP) 2003-365139

(51) Int. Cl. A61B 5/053

(52) U.S. Cl. 600/547

(57) ABSTRACT

The electrode supporting portion 11 is formed of the base 12 attached to the belt and the electrode portion 13 having the electrode 130. The pillar portion 132 of the electrode portion 13 is inserted into the hole 122 formed through the cover 121, and is oscillatably supported via the gap formed between the outer peripheral surface of the pillar portion 132 and the inner peripheral edge of the hole 122. There is provided the coiled spring 14 disposed between the hollow inside of the pillar portion 132 and the bottom plate 124, and the pressure sensing element 15 is provided in the center of the bottom plate 124 which receives the load of the end portion of the spring 14. The expansion and contraction of the spring 14 and the oscillation of the electrode portion 13 allows the electrode 130 to follow the movement of the body surface, thereby reducing changes in the contact condition between the electrode 130 and the subject's body.

### BACKGROUND OF THE INVENTION

1. Field of the Invention

The present invention relates to an impedance measuring device for measuring human body impedance, and a health care guideline advising device for providing guideline information useful for health care, such as body fat percentage.

2. Description of the Related Art

The technique to measure impedance by using a belt-type device is disclosed, e.g. in Japanese Patent Application Laid-open (JP-A) Nos. 11-113870 and 2001-252257.

### SUMMARY OF THE INVENTION

In these conventional arts, however, measurement is done by making a subject wear a belt-type device around his/her abdomen, so that the subject's abdomen circumference may change as he/she breathes, whereby the contact position between the electrode and the subject's body may be slightly shifted or the contact condition between them may be changed. Such a shift in the contact position of the electrode or change in the contact condition may cause a change in the impedance value to be obtained, thereby affecting measurement precision.

The present invention, which has been contrived to solve these conventional problems, has an object of providing an impedance measuring device capable of high precision measurement by stabilizing its contact condition, and also providing a health care guideline advising device.

In order to achieve the above object, the present invention is an impedance measuring device comprising: an electrode which is to get in contact with a subject's body; an electrode supporting portion for supporting the electrode; a belt-shaped member for supporting the electrode supporting portion and being worn on the subject's body, wherein the impedance measuring device comprises: pressing force generating unit to generate a pressing force against a surface of the subject's body which is in contact with the electrode; and pressing force detecting unit to detect the pressing force.

The impedance measuring device preferably further comprises contact condition determining unit determine a contact condition between the electrode and the surface of the subject's body, based on the detected pressing force.

The impedance measuring device preferably further comprises notifying unit to notify the subject of a contact error when the contact condition determining unit has determined the contact condition to be an error.

The impedance measuring device preferably further comprises notifying unit to notify the subject of an appropriate contact when the contact condition determining unit has determined the contact condition to be appropriate.

The impedance measuring device preferably further comprises fluctuation detecting and notifying unit to detect fluctuations in the pressing force with the pressing force detecting unit and then notifying the subject of detection of the fluctuations in the pressing force.

It is preferable that the impedance measurement gets started when the contact condition determining unit has determined the contact condition to be appropriate.

It is preferable that the electrode supporting portion supports the electrode in such a manner that the electrode is oscillatable with respect to the belt-shaped member.

It is preferable that the present invention is structured as a health care guideline advising device which is provided with the impedance measuring device, and which provides guideline information useful for health care, based on measured impedance.

The guideline information can be anything as long as it can be calculated from the measured impedance value, such as visceral fat quantity, body fat percentage or lean mass index, and these are not the only information obtainable by the device.

The present invention provides an impedance measuring device capable of high precision measurement by stabilizing its contact condition, and a health care guideline advising device.

### BRIEF DESCRIPTION OF THE DRAWINGS

FIG. 1A is a perspective view of an electrode supporting portion according to an embodiment of the present invention,

FIG. 1B is a cross sectional perspective view of the electrode supporting portion of the embodiment of the present invention, and

FIG. 1C is a cross sectional view of the electrode supporting portion of the embodiment of the present invention.

FIG. 2 is a block diagram showing the schematic structure of a visceral fat meter according to the embodiment of the present invention.

FIG. 3 is a view showing the whole outer structure of the visceral fat meter according to the embodiment of the present invention.

FIG. 4 is a flowchart showing a processing procedure in the visceral fat meter according to the embodiment of the present invention.

FIG. 5 is a flowchart showing another processing procedure in the visceral fat meter according to the embodiment of the present invention.

### DESCRIPTION OF THE PREFERRED EMBODIMENTS

The following is a description of an embodiment of the present invention with reference to drawings.

The visceral fat meter 1 as the health care guideline advising device according to the embodiment of the present invention is formed of a belt which is to be worn around a subject's abdomen and an electrode which is to get in contact with his/her skin.

(Structure of the Electrode Supporting Portion)

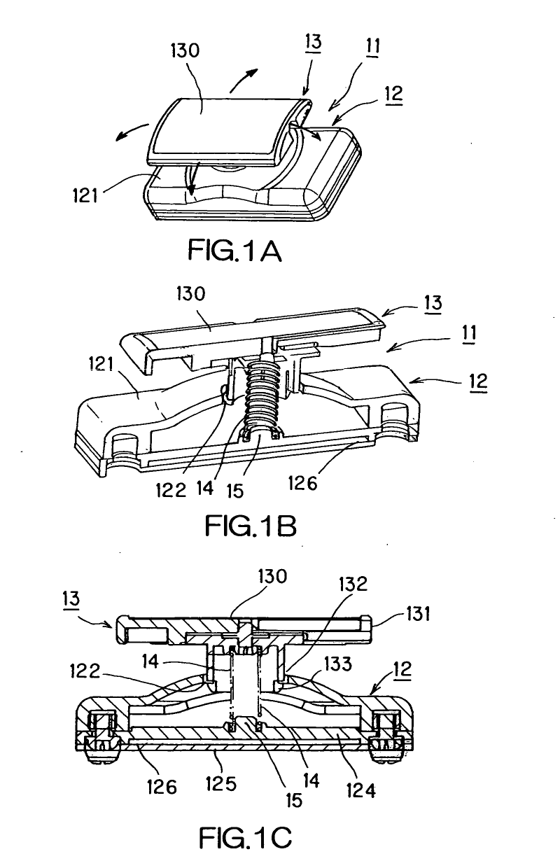

FIG. 1A is a perspective view showing the outer appearance of the electrode supporting portion 11 attached to the belt (belt-shaped member) to be worn around a subject's abdomen, FIG. 1B is a cross sectional perspective view of the electrode supporting portion 11, and FIG. 1C is a cross sectional view of the electrode supporting portion 11.

The electrode supporting portion 11 is mainly formed of a base 12 to be attached to the belt, and an electrode portion 13 movable with respect to the base 12.

The base 12 is substantially rectangular and flat along the belt. The base 12 has a cover 121 whose top surface swells in the center like a dome, and on the swollen center is provided a hole 122 into which the electrode portion 13 is fixed.

The base 12 is formed of a bottom plate 124 and a tabular attachment member 125 which are laminated on the belt side. Between the bottom plate 124 and the tabular attachment member 125 is provided a gap 126 for passing the belt through, and the attachment member 125 is fixed on the bottom plate 124 with the belt therebetween. The base 12 can be either fixed on a predetermined position or designed to be movable along the belt.

The cover 121 is hollow on the bottom plate side, and the edges of the cover 121 are screwed into the bottom plate 124 and the attachment member 125.

An electrode 130 is disposed on the top of a head portion 131 of the electrode portion 13 which is substantially shaped like a letter T. The electrode 130 is curved in the direction in which the belt is extended, and is shaped like a rectangle which has a curved surface and whose long side is orthogonal to the belt. The electrode portion 13 has, on the base 12 side, a cylindrical pillar portion 132 extending orthogonal to the head portion 131. The pillar portion 132 is inserted into the hole 122 formed in the cover 121 of the base 12, and is supported via a gap formed between the outer surface of the pillar portion 132 and the inner peripheral edge of the hole 122. At the end of the pillar portion 132 on the bottom plate side is provided a flange portion 133 extending to the outer diameter side. The flange portion 133 is engaged with the inner peripheral edge of the hole 122, thereby functioning as a stopper for preventing the electrode portion 13 from pulling out of the hole 122. The pillar portion 132 is shaped like a cylinder which is open on the bottom plate side and hollow inside, and one end of a coiled spring (pressing force generating unit) 14 is held in the hollow inside of the pillar portion 132. The other end of the coiled spring 14 is fixed on the bottom plate 124.

In the center of the bottom plate 124 which receives the load of the end portion of the spring 14 is provided a pressure sensing element (pressing force detecting unit) 15 for detecting the pressing force with which the electrode 130 disposed on the head portion 131 of the electrode portion 13 is pressed. The elasticity of the spring 14 energizes the electrode 130 in the direction departing from the bottom plate 124 (towards the body surface of the subject). However, the electrode portion 13 is locked by the cover 121 as a result of the engagement between the flange portion 133 and the inner peripheral edge of the hole 122. The presence of the gap between the pillar portion 132 and the hole 122 allows the electrode portion 13 to oscillate in the direction orthogonal to the axis of the pillar portion 132.

Therefore, even if there is a relative positional change in the direction orthogonal to the body surface due to the subject's breathing or the like, the expansion and contraction of the spring 14 allows the electrode 130 to follow the movement of the body surface, thereby reducing changes in the contact condition between the electrode 130 and the subject's body. Even if a body movement or a belt movement causes a relative positional change in the direction parallel to the body surface, the pillar portion 132 oscillates with respect to the base 12, so that the electrode 130 can follow a body surface movement, thereby reducing changes in the contact condition between the electrode 130 and the subject's body.

Although FIG. 1 does not show a structure for applying or taking out signals to or from the electrode 130, it is possible to use, e.g. a structure in which a tongue piece extended from the electrode 130 having the curved surface is projected into the pillar portion 132, and connected with an extension line.

In the aforementioned embodiment, the electrode 130 is a rectangle with a curved surface; however, it can be of other shapes such as a semisphere. The shapes of the electrodes in respective electrode supporting portions 11 can be changed in accordance with sites where the electrode supporting portions 11 are disposed, that is, in accordance with the shape of the body portion which is to get in contact with the electrodes.

In the aforementioned embodiment, the pressure sensing element 15 is disposed in the load receiving portion of the spring 14; however, the pressure sensing element 15 is not the only means usable as the pressing force detecting unit to directly or indirectly detect the pressing force against the electrode.

In the aforementioned embodiment, the pressing force of the electrode 130 against the subject's body is generated by the coiled spring 14; however, the pressing force generating unit can be other elastic members such as sponge or rubber.

(Structure of the Visceral Fat Meter)

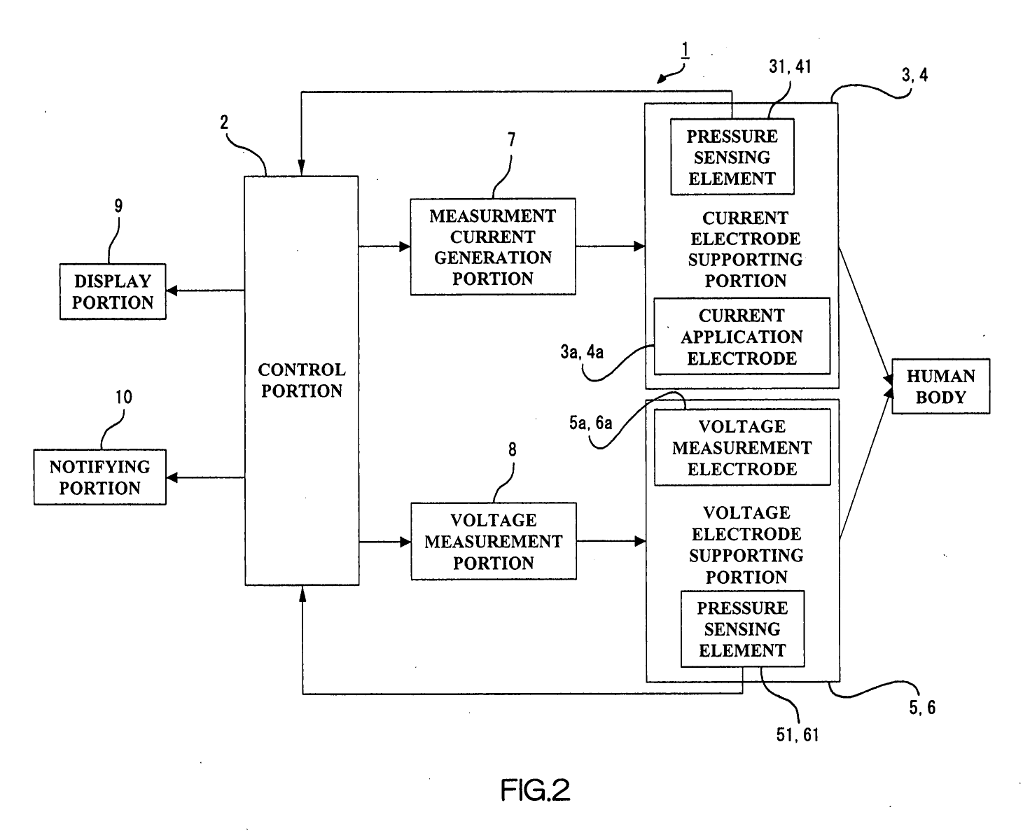

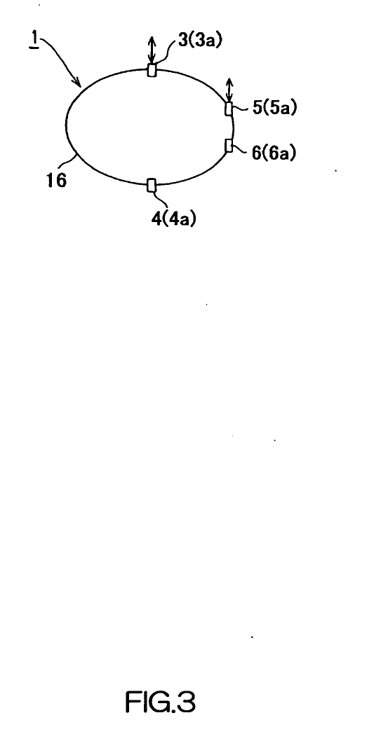

FIG. 2 is a block diagram showing the schematic structure of the visceral fat meter 1. FIG. 3 is a view showing the whole outer structure of the visceral fat meter 1. As a method for calculating health care guideline information such as visceral fat quantity or body fat percentage by using measured impedance values in the visceral fat meter according to the embodiment, well-known methods can be used; however, they are not the only methods usable in this case.

The visceral fat meter 1 is mainly formed of a control portion 2, current electrode supporting portions 3, 4, current application electrodes 3a, 4a, voltage electrode supporting portions 5, 6, voltage measurement electrodes 5a, 6a, a measurement current generation portion 7, a voltage measurement portion 8, pressure sensing elements 31, 41, 51 and 61, a display portion 9 and notifying portion 10.

The aforementioned electrode supporting portion 11 can be applied to any of the current electrode supporting portions 3, 4 and voltage electrode supporting portions 5, 6 in the visceral fat meter 1 shown in FIG. 2. In that case, the electrode 130 corresponds to each of the current application electrodes 3a, 4a and voltage measurement electrodes 5a, 6a.

The control portion 2, which is formed of a CPU, a ROM, a RAM or the like, performs a calculation process or controls each portion of the device. The control portion 2 directs the operation of the measurement current generation portion 7 so as to apply a high-frequency current to the subject's body via the current application electrodes 3a, 4a provided in the current electrode supporting portions 3, 4. Apotential caused by the resistance of the subject's body against the applied current is detected via the voltage measurement electrodes 5a, 6a provided in the voltage electrode supporting portions 5, 6. Output signals of the voltage electrode supporting portions 5, 6 are transmitted to the voltage measurement portion 8 to measure voltage values. The voltage values measured in the voltage measurement portion 8 are taken into the control portion 2, and subjected to predetermined calculation processes to find guideline information useful for health care, such as measured impedance value or body fat percentage. The health care guideline information such as measured impedance value or body fat percentage is displayed on the display portion 9. The notifying portion 10, as will be described later, notifies the subject about the occurrence of malfunction and the like by way of audio or visual information. As described above, the current electrode supporting portions 3, 4 and the voltage electrode supporting portions 5, 6 are provided with pressure sensing elements 31, 41, 51 and 61 for detecting pressing forces exerted on the electrodes, so that outputs of the pressure sensing elements 31, 41, 51 and 61 can be taken into the control portion 2.

The visceral fat meter 1 includes a belt 16 to be worn around the subject's abdomen, and current electrode supporting portions 3, 4 equipped with current application electrodes 3a, 4a and voltage electrode supporting portions 5, 6 equipped with voltage measurement electrodes 5a, 6a which are to get in contact with the subject's abdomen surface. The current application electrodes 3a, 4a are supposed to get in contact with the front and back surfaces of the subject's abdomen, and the voltage measurement electrodes 5a, 6a are supposed to get in contact with the front and back portions on the subject's right side.

Here, the control portion 2 and the pressure sensing elements 31, 41, 51, and 61 compose contact condition determining unit.

The length of the belt 16 can be adjusted to the subject's abdomen circumference.

(Behavior of the Visceral Fat Meter)

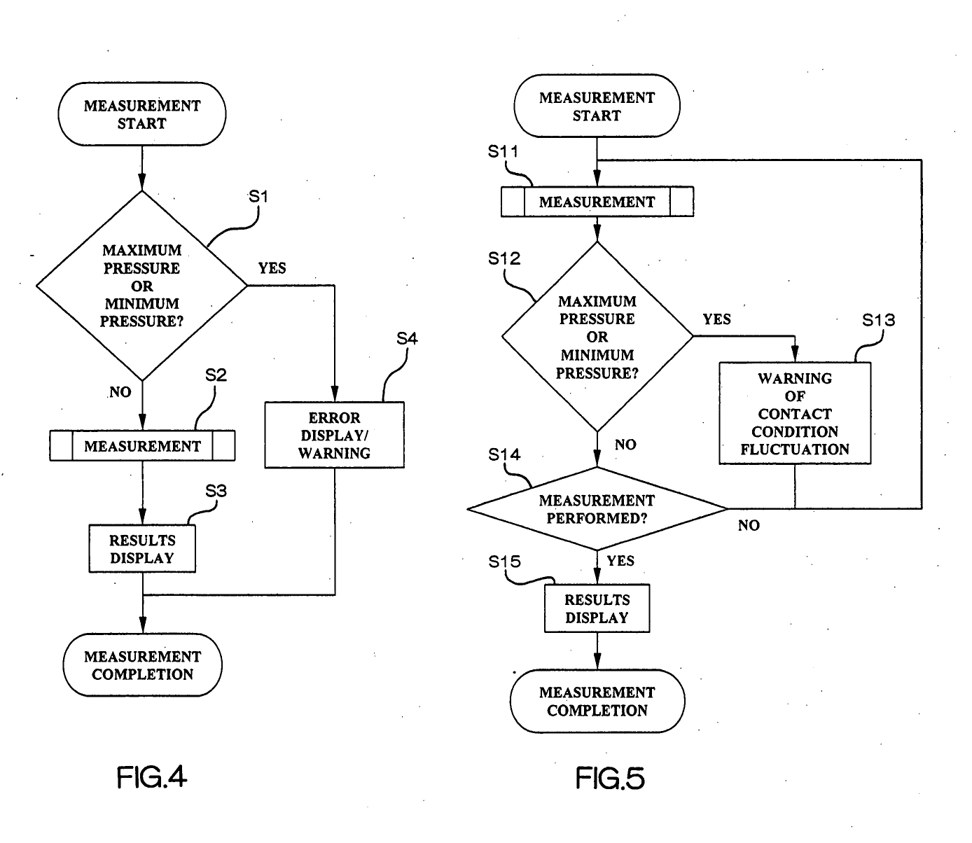

With reference to the flowchart shown in FIG. 4, a measuring process on the visceral fat meter 1 according to the embodiment of the present invention will be described as follows.

When the measurement start is directed, it is determined whether the output of the pressure sensing element indicates a maximum pressure or a minimum pressure (Step 1). When exceeding a predetermined upper limit, the pressing force obtained from the output of the pressure sensing element is determined to be the maximum pressure, and when below a predetermined lower limit, it is determined to be the minimum pressure.

In Step 1, when the pressing force is determined to be neither the maximum pressure nor the minimum pressure, an impedance measurement is performed (Step 2) , and then calculation results of visceral fat quantity, body fat percentage or the like are displayed on the display portion 9 (Step 3), thereby terminating the measurement.

In Step 1, when the pressing force has been determined to be either the maximum pressure or the minimum pressure, the display portion 9 performs an error display or the notifying portion 10 gives a warning (Step 4), thereby terminating the measurement.

When the belt is not worn tight enough, the contact condition between the subject's body and the electrodes becomes unstable, thereby decreasing the measuring precision. However, in the visceral fat meter according to the present embodiment, the output of the pressure sensing element is determined to demonstrate the minimum pressure and then no measurement is performed so as to avoid low precision measurement. On the other hand, when the belt is worn too tight, the pressing force of the electrodes cause changes in the measuring target condition, such as the position of abdomen fat or the abdomen circumference, thereby making accurate measurement difficult. In the visceral fat meter according to the present embodiment, however, the output of the pressure sensing element is determined to demonstrate the maximum pressure, and then no measurement is performed so as to avoid low precision measurement.

When the belt is not worn appropriately like this, either an error display is shown or a warning is offered so as to provide the subject with an appropriate wearing condition.

During the measurement at Step 2, it is possible to make either the display portion 9 show or the notifying portion 10 notify that the belt's tightness is appropriate.

FIG. 5 is a flowchart showing another measuring procedure on the visceral fat meter according to the embodiment of the present invention.

When the measurement start is directed, measurement gets started (Step 11).

Then, the presence or absence of fluctuations in the pressing force is determined from the output of the pressure sensing element (Step 12). When there are fluctuations in the pressing force in Step 12, it is regarded as the presence of fluctuations in the contact condition between the electrode and the subject's body, so that a warning is offered (Step 13), and the process returns to Step 11. When there are no fluctuations in pressing force in Step 12, whether the measurement is performed or not is determined (Step 14). When the measurement is not complete in Step 14, the process returns to Step 11. When the measurement is complete in Step 14, calculation results of visceral fat quantity, body fat percentage or the like are displayed on the display portion 9 (Step 15), thereby terminating the measurement.

The fluctuations which occur in the contact condition between the electrode and the subject's body and whose presence or absence is determined in Step 12 may result from changes in the posture of the subject or his/her breathing. With the aforementioned procedure, in a case that the fluctuations in the contact condition between the electrode and the subject's body may cause fluctuations in the measuring requirements, a warning is offered so that the subject can find a desired measuring posture and breathing condition.

In the aforementioned procedure, when fluctuations in the contact condition between the electrode and the subject's body are detected, a warning display is repeated. Instead of this, it is possible to perform an error display after a warning display is repeated a predetermined number of times and then terminate the measurement.

It is also possible to make either the display portion 9 show or the notifying portion 10 notify the detection of fluctuations in the pressing force during measurement.

When using the visceral fat meter according to the embodiment for the first time, the subject cannot recognize the appropriate tightness to wear the belt. Therefore, he/she can learn the tightening force (wearing condition) suitable to himself/herself by operating the device in accordance with the flowchart of FIG. 4. From the second time on, the subject can follow the flowchart of FIG. 5 because the belt length does not change largely day by day.

1. An impedance measuring device comprising:
an electrode which is to get in contact with a subject's body;
an electrode supporting portion for supporting the electrode;
a belt-shaped member for supporting the electrode supporting portion and being worn on the subject's body, wherein
the impedance measuring device comprises:
pressing force generating unit to generate a pressing force against a surface of the subject's body which is in contact with the electrode; and
pressing force detecting unit to detect the pressing force.

2. An impedance measuring device according to claim 1 further comprising:
contact condition determining unit to determine a contact condition between the electrode and the surface of the subject's body, based on the detected pressing force.

3. An impedance measuring device according to claim 2 further comprising:
notifying unit to notify the subject of a contact error when the contact condition determining unit has determined the contact condition to be an error.

4. An impedance measuring device according to claim 2 further comprising:
notifying unit to notify the subject of an appropriate contact when the contact condition determining unit has determined the contact condition to be appropriate.

5. An impedance measuring device according to any one of claims 1 to 4 further comprising:
fluctuation detecting and notifying unit detect fluctuations in the pressing force with the pressing force detecting unit and then notifying the subject of detection of the fluctuations in the pressing force.

6. An impedance measuring device according to any one of claims 2 to 4, wherein an impedance measurement gets started when the contact condition determining unit has determined the contact condition to be appropriate.

7. An impedance measuring device according to any one of claims 1 to 6, wherein the electrode supporting portion supports the electrode in such a manner that the electrode is oscillatable with respect to the belt-shaped member.

8. A health care guideline advising device which is provided with the impedance measuring device according to any one of claims 1 to 7, and which provides guideline information useful for health care, based on measured impedance.
